(19) United States
(12) Patent Application Publication
(10) Pub. No.: US 2026/0216042 A1
(43) Pub. Date: Jul. 30, 2026

(54) ENZYME COMPOSITION FOR TREATING DENTAL PLAQUE AND PLAQUE BACTERIAL BIOFILMS, AND FOR THE NATURAL WHITENING OF TOOTH ENAMEL

(71) Applicant: Jean Dominique DANA, DREVANT (FR)

(72) Inventors: Jean Dominique DANA, DREVANT (FR); Lou DANA, PARIS (FR)

(21) Appl. No.: 19/152,345

(22) PCT Filed: Jan. 30, 2024
(86) PCT No.: PCT/EP2024/052264

§ 371 Date: Jul. 30, 2025

(30) Foreign Application Priority Data
Feb. 3, 2023 (FR) FR2301034

(51) Int. Cl. A61K 8/66; A61K 8/25; A61K 8/27; A61K 8/60; A61K 8/64; A61Q 11/00

(52) U.S. Cl. CPC A61K 8/66; A61K 8/25; A61K 8/27; A61K 8/60; A61K 8/64; A61Q 11/00; C12Y 101/03004; C12Y 111/01007; C12Y 302/01003; C12Y 302/0101; C12Y 302/01011; C12Y 302/01059

(57) ABSTRACT

An oral health enzyme complex for use in treating dental plaque and dental plaque bacterial biofilms and for the natural whitening of enamel, the complex including a combination of the following compounds a sucrase enzyme, in particular capable of hydrolysing the P-2,6 and/or P-2, I bonds of sucrose; a mixture of glucanohydrolase enzymes comprising a mutanase having exolytic activity and obtained from a fungus, and a mutanase having endolytic activity and obtained from a bacterium, in particular capable of hydrolysing (1-3)-a-D-glucans and (1-6)-a-D-glucans; a glycoside hydrolase enzyme, in particular capable of hydrolysing the (1-6)-a-D glycosidic bonds of monosaccharide dimers and trimers; an oxide reductase enzyme, in particular capable of forming hydrogen peroxide from glucose and monosaccharide monomers; a peroxidase enzyme, in particular capable of converting thiocyanate into hypothiocyanate in the presence of hydrogen peroxide.

### TECHNICAL FIELD OF THE INVENTION

The technical field of the invention is that of bucco-dental hygiene and cosmetic products.

The present invention relates to a new enzyme complex able to prevent formation of dental plaque, caries and tartar, while making the enamel naturally whiter. The present invention also relates to a new form of prevention and treatment of the dental bacterial biofilm by said enzyme complex.

### TECHNOLOGICAL BACKGROUND OF THE INVENTION

Sugar from food, which is more than 90% sucrose, is degraded into glucans by cariogenic bacteria in the buccal cavity, which form a bacterial biofilm on the tooth enamel. A bacterial biofilm is an organised and structured community of bacterial cells entangled in an extracellular matrix of variable density and composition. The major part of the dental biofilm matrix is made up of polysaccharides. Indeed, up to 40% of the dry weight of dental biofilm is comprised of polysaccharides. In the presence of sucrose, this bacterial biofilm grows thicker on the teeth, first forming soft dental plaque, comprised of soluble glucans (known as (1-6)-α-D-glucan or dextrans), and then hard dental plaque formed by the transformation of these dextrans into water-insoluble glucans (known as (1-3)-α-D-glucan or mutans).

In the case of a predominantly sugary diet, the pH of dental plaque becomes acidic and promotes formation of caries, while in the case of a predominantly meat-based diet, the pH becomes alkaline and the hard plaque calcifies to form tartar, which is very difficult to remove.

According to a 2021 WHO report, in 2020, 3.5 billion people worldwide suffered from dental problems related to the formation of caries and/or tartar. Likewise, according to data from the American National Health and Nutrition Examination Survey in 2018, 59% of adolescents aged 12 to 19 and 92% of adult aged 20 to 64 suffered from dental caries in their permanent teeth. There is therefore still a need to reduce these phenomena.

Current approaches to reducing dental plaque include mechanical cleaning, chemicals, fluoride applications and broad-spectrum antiseptic antibacterial agents such as chlorhexidine. Although effective in the short term, these therapies all have side effects. Indeed, mechanical cleaning is known to cause tooth sensitivity and gum bleeding problems, chemicals are known to represent a health risk, the use of fluoride can cause major adverse effects on human health, while having only a modest effect on preventing dental caries, and chlorhexidine can cause tooth staining and shutter caries. Furthermore, the use of antiseptics and chemicals has been shown to disrupt the commensal microbiota, increasing the pathogenic characteristics and cytotoxicity of host cells and causing other adverse effects on bucco-dental health.

These solutions are therefore unsatisfactory.

There is a growing preference for more preventive treatments that are more respectful of the overall health of the consumer, with minimal side effects, such as the use of probiotics, prebiotics or natural substances, but their therapeutic usefulness is notoriously limited.

Among the pathogenic bacteria in the buccal cavity, Streptococcus mutans is primarily responsible for the formation of bacterial biofilm, the main cause of caries, as it uses dietary sucrose to produce lactic acid and synthesise extracellular polysaccharides, in particular (1-3) and (1-6)-α-D-glucans, which promote biofilm formation and increase the resistance of microorganisms in the biofilm. Lactic acid demineralises the tooth surface, promoting formation of dental plaque and periodontitis. The acidic biofilm formed by Streptococcus mutans is highly resistant to the hostile environment, host immunity and antimicrobial treatments, especially lysozyme. The same research on dental pathology shows the difficulty of eliminating these streptococci protected within the matrix by the bacterial biofilm.

There is therefore a need to prevent the proliferation of Streptococcus mutans.

Lactoperoxidase is an enzyme known for its bactericidal effect in the presence of hydrogen peroxide and thiocyanate, especially against Streptococcus mutans. It is known to provide these three substances by supplying exogenous lactoperoxidase, glucose oxidase and potassium thiocyanate. In this type of composition, however, the formation of hydrogen peroxide is insufficient for the bactericidal effect of lactoperoxidase to significantly affect the Streptococcus mutans biofilm in terms of reducing caries and/or tartar formation. Further, the addition of exogenous glucose to such compositions is not recommended in the field of bucco-dental hygiene. This solution is therefore not satisfactory for preventing the proliferation of Streptococcus mutans.

In general, the major problem in the fight against caries and tartar, and in preserving healthy and beautiful teeth, is therefore to have dental plaque with a neutral pH that is as physiological as possible on a daily basis. Recent research on dental diseases shows that the best means of preventing them is to be able to eradicate bacterial biofilms. To this end, it is especially appropriate to prevent the formation of Streptococcus mutans biofilm and to break down existing biofilms in order to replace them with physiological plaque, i.e. the natural protective biofilm that surrounds healthy teeth and is made up of saliva and microbiota of the buccal cavity.

In addition to having healthy teeth, it is also desirable to have teeth that are as beautiful as possible. There is therefore a constant need to whiten tooth enamel in order to combat yellowing, which is caused especially by ageing and the formation of insoluble dental plaque.

Documents FR 2651433, FR 2822700, FR 3020758, FR 2803199 and WO 2021/144211 each disclose an enzyme complex for bucco-dental health, but none of these documents disclose a mixture of glucanohydrolase enzymes comprising a mutanase having exolytic activity and obtained from a fungus, and a mutanase having endolytic activity and obtained from a bacterium.

### SUMMARY OF THE INVENTION

The invention offers a solution to the problems previously discussed, by providing an enzyme complex whose composition, which is based especially on the synergistic action of its different constituents, results from numerous studies carried out by the applicant. Being of natural origin, this enzyme complex is respectful of the overall health of the consumer and has no side effects.

One aspect of the invention relates to an enzyme complex for use in the treatment of dental plaque and bacterial biofilm of the plaque and for the natural whitening of tooth enamel, comprising the following compounds:
a sucrase enzyme,
a mixture of glucanohydrolase enzymes comprising a dextranase, a mutanase having exolytic activity and obtained from a fungus, and a mutanase having endolytic activity and obtained from a bacterium,
a glycoside hydrolase enzyme,
an oxidoreductase enzyme,
a peroxidase enzyme.

According to one aspect of the invention, the mixture of glucanohydrolase enzymes comprises a mixture of chimeric glucanases comprising a dextranase and a mutanase bonded by genetic engineering.

According to another aspect of the invention, the mutanase having exolytic activity is obtained from a Trichoderma Harzianum fungus.

According to a further aspect of the invention, the mutanase having endolytic activity is obtained from a Paracoccus mutanolyticus, Paenibacillus or Streptomyces bacterium.

According to one aspect of the invention, the sucrase enzyme is an isomaltase enzyme or an invertase enzyme.

According to another aspect of the invention, the glycoside hydrolase enzyme is an amyloglucosidase enzyme, preferably an amyl-α-1,6 glucosidase.

According to a further aspect of the invention, the oxidoreductase enzyme is a glucose oxidase.

According to one aspect of the invention, the peroxidase enzyme is a lactoperoxidase.

According to another aspect of the invention, the enzyme complex comprises the following compounds:
a sucrase, preferably an invertase or an isomaltase,
a dextranase,
a mixture of mutanases comprising a mutanase having exolytic activity and obtained from a fungus, and a mutanase having endolytic activity and obtained from a bacterium,
an amyloglucosidase, preferably an amyl-α-1,6 glucosidase,
a glucose oxidase, and
a lactoperoxidase.

According to a further aspect of the invention, the enzyme complex comprises the following compounds, the percentages indicated being percentages by volume:
10 to 30%, preferably 15 to 25%, of a sucrase,
1 to 10%, preferably 4 to 7%, of dextranase,
10 to 40%, preferably 15 to 30%, of a mixture comprising a mutanase having exolytic activity and obtained from a fungus and a mutanase having endolytic activity and obtained from a bacterium,
20 to 60%, preferably 30 to 50%, of an amyloglucosidase, preferably an amyl-α-1,6 glucosidase,
5 to 20%, preferably 7 to 15%, of glucose oxidase, and
1 to 10%, preferably 4 to 7%, of lactoperoxidase.

According to one aspect of the invention, the enzyme complex further comprises lysozyme.

Another aspect of the invention relates to a buccal composition for use in the treatment of dental plaque and bacterial biofilm of the plaque and the natural whitening of tooth enamel, which comprises an enzyme complex as previously described.

According to one aspect of the invention, the buccal composition comprises 1.4 to 3%, preferably 2.0 to 2.5%, of enzyme complex.

According to another aspect of the invention, the buccal composition further comprises lactoferrin.

According to a further aspect of the invention, the buccal composition further comprises:
20 to 60%, preferably 30 to 50%, of sorbitol,
1 to 20%, preferably 5 to 10%, of xylitol,
0 to 3%, preferably 1 to 2%, of polysorbate 80,
0 to 3%, preferably 1 to 2%, of polysorbate 20,
0 to 0.5%, preferably 0.1 to 0.2%, of sodium saccharin,
0 to 5%, preferably 1 to 2%, of colloidal silica,
0 to 3%, preferably 1 to 2%, of dimethicone,
0 to 5%, preferably 1 to 2%, of mint flavouring and/or mint essential oils,
0.1 to 0.5%, preferably 0.1 to 0.3%, of zinc citrate,
0 to 0.5%, preferably 0.1 to 0.2%, of potassium thiocyanate,
0 to 0.5%, preferably 0.1 to 0.2%, of preservative, and
water, in sufficient quantity to make up 100%.

According to one aspect of the invention, the buccal composition is in the form of a paste, liquid, gel, mouthwash, mist, spray, capsule, tablet or chewing gum.

As will be seen in detail hereinafter, by combining various bacteriostatic and bactericidal actions against cariogenic streptococci, the glycolytic enzyme system of the invention makes it possible to significantly reduce the toxicity of Streptococcus mutans. This enzyme complex acts on dietary sucrose in such a way as to deprive the bacteria of their substrate, but also by directly degrading the cell wall of these streptococci.

By virtue of the sucrase enzyme, preferably an invertase or an isomaltase, which hydrolyses the β-2,6 and/or β-2,1 bonds of sucrose, the enzyme complex according to the invention advantageously reduces the amount of sucrose available in the buccal cavity by converting it into monomers of monosaccharide (glucose) and fructose, which deprives cariogenic bacteria of metabolic substrate, greatly reduces the production of soluble and insoluble glucans, and thus contributes to the reduction of bacterial biofilm.

By virtue of the mixture of glucanohydrolase enzymes, which hydrolyse the (1-3) and (1-6)-α-D-glucan bonds, which are key structural and functional components of the biofilm matrix of Streptococcus mutans, the enzyme complex according to the invention advantageously prevents the formation of bacterial biofilm and disassembles existing biofilms. The destruction of the bacterial biofilm further reduces the formation of dental plaque, which is malodorous and yellows the teeth under the influence of tobacco or colouring agents such as coffee or tea. The soluble glucans are converted into monosaccharide dimers or trimers by the mixture of glucanohydrolase enzymes, which are then converted into monosaccharide monomers by the action of the glycoside hydrolase enzyme, preferably amyloglucosidase, for example amyl-α-1,6 glucosidase, which hydrolyses the (1-6)-α-D oside bonds of the monosaccharide dimers and trimers.

The preferential combination of a dextranase with a mutanase, more preferably from different fungi or bacteria, or a chimeric glucanase (dextranase-mutanase), shows very satisfactory effects in preventing the formation of Streptococcus mutans biofilm and in disassembling existing biofilms in a few minutes at nanomolar concentrations. This combination significantly improves sensitivity of the biofilm to antimicrobial and antibacterial agents, such as lysozyme, highlighting its great potential in fighting against acid biofilm-related dental caries.

The residual osidic derivatives resulting from the degradation of glucans in dental plaque are then converted into monosaccharide monomers by a glycoside hydrolase enzyme, which hydrolyses (1-6)-α-D osides, while the monosaccharide monomers are converted by the oxidoreductase enzyme into D-glucono-delta-lactone, but also, and more importantly, into hydrogen peroxide. Indeed, the oxidoreductase enzyme forms hydrogen peroxide from the monosaccharide monomers. The formation of hydrogen peroxide is advantageous because it has an antiseptic effect that actively participates in eliminating Streptococcus mutans, and a natural whitening effect on enamel, which contributes to a more beautiful appearance of the teeth.

These beneficial effects are advantageously enhanced by the fact that the enzyme complex according to the invention provides several sources of hydrogen peroxide in the buccal cavity through the in situ conversion of sucrose, monosaccharide dimers and trimers, soluble glucans and insoluble glucans into monosaccharide monomers, i.e. glucose. Glucose thus regularly available is converted by the gluco-oxidase enzyme to produce hydrogen peroxide directly in contact with the teeth in a regular, gentle and lasting manner, for an optimal effect on oral hygiene and enamel whitening. As glucose is converted in real time, the amount of glucose present in the buccal cavity at any given time remains advantageously much lower and therefore much less harmful than when exogenous glucose is added.

Hydrogen peroxide generated in situ by virtue of the enzyme complex according to the invention is produced in much smaller amounts than those usually used for tooth whitening, which are supplied exogenously, but it is also present in the buccal cavity for a longer period of time, which allows a satisfactory progressive whitening effect to be achieved without the usual effects associated with a massive exogenous supply of hydrogen peroxide, namely: demineralisation of hard tooth tissue, irritation of the mucous membranes, reaction with shutter materials, premature wear of the enamel with hypersensitivity and weakening of the teeth.

Further, in the presence of hydrogen peroxide, the peroxidase enzyme advantageously converts thiocyanate naturally present in saliva into hypothiocyanate, which is a powerful bactericide and further enhances action of the enzyme complex of the invention with respect to dental health. To enhance this effect, it is also possible to add exogenous thiocyanate.

The addition of a sucrase, preferably an invertase or an isomaltase, to the enzyme complex according to the invention is advantageous in that this enzyme breaks down any starch and glycogen present in the buccal cavity into monosaccharide dimers (maltose), which are then converted into monosaccharide monomers by an alpha-amylase naturally present in saliva.

The addition of lysozyme to the enzyme complex according to the invention is advantageous in that it hydrolyses the polysaccharide cell wall of bacteria and, in the absence of the bacterial biofilm generated by Streptococcus mutans, exhibits strong bactericidal activity, actively participating in bucco-dental hygiene. Indeed, the bactericidal activity of the enzyme complex according to the invention is enhanced by lysozyme, which acts more effectively “after” the disappearance of the bacterial biofilm.

In summary, through the synergistic action of its components, the enzyme complex according to the invention provides the following beneficial effects while respecting the overall health of the consumer and without side effects:
anti-cariogenic sugar action by inhibiting the acid conversion of dietary sugars and thus a preventive effect against caries,
a destructuring action on the bacterial biofilm of dental plaque and therefore a preventive effect against periodontal disease and tartar,
a sanitising action on the microbiota of the buccal cavity by reducing pathogenic bacteria under the stimulating effect of the salivary lactoperoxidase system,
a gentle and prolonged whitening action on tooth enamel through the natural production of hydrogen peroxide.

The addition of lactoferrin in a composition for buccal use comprising an enzyme complex, by ensuring the chelation of iron necessary for the binding of the polysaccharides of the microbial envelope, necessary for the survival of cariogenic streptococci, advantageously leads to better eradication of the Streptococcus mutans biofilm and participates in the synergy discussed above. Indeed, lactoferrin lyses the bacterial membrane of many organisms, including acidogenic streptococci, and thus greatly increases the body's immune defences.

The invention and its different applications will be better understood upon reading the following description.

### DETAILED DESCRIPTION

Streptococcus mutans uses dietary sucrose to synthesise extracellular polysaccharides (EPS) in order to promote formation of bacterial biofilms. Recent studies show that the EPS matrix, consisting of a glucose polymer bonded to α-(1-6) (dextran) with α-(1-3) (mutane) branch bonds, plays a crucial role in regulating formation and virulence of cariogenic biofilm by influencing the physical and biochemical properties of the biofilm. The cariogenic biofilm can promote accumulation and adhesion of microorganisms and accelerate the cohesion of bacterial cells between themselves and with the apatite surface, thus modulating the initial steps of cariogenic biofilm development and proliferation and facilitating the formation of mature dental plaque. In addition, as a reserve energy source, the EPS matrix protects microorganisms from hostile influences, affects the diffusion of substances into and out of the biofilm, and helps to concentrate metal ions and other physiological nutrients in a microenvironment. As a physical barrier, the cariogenic biofilm hinders the diffusion of antibiotics and host-induced antimicrobial factors into the deepest layers of the biofilm, thereby increasing resistance of the microorganisms in the biofilm. To do this, Streptococcus mutans produces a dextranase, DexA, which hydrolyses the α-1,6 bonds of dextran and produces isomaltoligosaccharides of different sizes. Dextranase DexA is crucial in the bacterial biofilm formation process and is considered responsible for the pathogenic dental plaque ecosystem: DexA dextranase hydrolyses glucans as potential storage polysaccharides to provide nutrients for bacterial metabolism and controls the amount and content of extracellular glucans to make it more adhesive. However, the effect of DexA dextranase can be counteracted by an externally supplied dextranase enzyme.

In studies made to select the appropriate enzyme complex according to the invention, it was especially sought to identify enzymes that selectively target and degrade EPS. In this study, it was noticed that dextranase shows much higher activity in inhibiting biofilm formation and disrupting a preformed biofilm when it is exogenously supplied in a combination with a mutanase. But even more interestingly, it was noticed that the use of mutanase from different sources, one of fungal origin with exolytic action, the other bacterial with endolytic action such as streptococcus, or a chimeric glucanase (dextranase-mutanase), prevents the formation of Streptococcus mutans biofilm in a very satisfactory manner and disassembles existing biofilms within minutes at nanomolar concentrations. This combination dramatically improves sensitivity of the biofilm to antimicrobial agents and lysozyme, highlighting its great potential in fighting against acid biofilm-related dental caries.

This innovative action on bacterial biofilm, combined with invertase, isomaltase or another enzyme from the sucrase family, which degrades sucrose and thus deprives cariogenic bacteria of their metabolic substrate, greatly reduces glucan production and thus contributes to a significant reduction in bacterial biofilm. The oside derivatives thus obtained give rise to monosaccharide monomers which, under the action of oxidoreductase enzymes, produce hydrogen peroxide, which triggers the highly bactericidal salivary lactoperoxidase reaction and leads to the disappearance of the bacterial biofilm.

In addition, this hydrogen peroxide, produced in small amounts but continuously, allows the tooth enamel to be whitened naturally and gradually, thus avoiding any problems of tooth sensitivity.

The enzyme complex according to the invention will be described in detail hereinafter.

By enzyme complex, it is meant here a stable association, according to a defined structure, of a number of different proteins whose various enzymatic activities contribute in an orderly manner to the generation of a synergistic activity on a target substrate.

The enzyme complex according to the invention is intended for buccal use to improve oral health and whiten tooth enamel.

It comprises a combination of the following components:
a sucrase enzyme, especially able to hydrolyse the β-2,6 and/or β-2,1 bonds of sucrose,
a mixture of glucanohydrolase enzymes, especially capable of hydrolysing (1-3)-α-D-glucans and (1-6)-α-D-glucans,
a glycoside hydrolase enzyme, especially capable of hydrolysing the (1-6)-α-D osidic bonds of monosaccharide dimers and trimers,
an oxidoreductase enzyme, especially capable of decomposing monosaccharide monomers to form hydrogen peroxide,
a peroxidase enzyme, especially capable of converting thiocyanate into hypothiocyanate in the presence of hydrogen peroxide.

The sucrase enzyme capable of hydrolysing the β-2,6 and/or β-2,1 bonds of sucrose is preferably an invertase or an isomaltase, which degrades sucrose into glucose and fructose.

The mixture of glucanohydrolase enzymes degrades dental plaque into dimers or trimers of monosaccharides. This is preferably a mixture of mutanase and dextranase. Dextranase degrades soft dental plaque by converting soluble dextrans into osidic polymers of the 1,6 monosaccharide dimer or trimer type, while mutanase degrades insoluble mutans in hard plaque by converting them into osidic polymers of the 1,3 monosaccharide dimer or trimer type.

In the mixture of glucanohydrolase enzymes, a mutanase having exolytic activity and a mutanase having endolytic activity are preferred. As a mutanase having exolytic activity, a mutanase obtained from a fungus, especially the Trichoderma Harzianum fungus, can be used. As a mutanase having endolytic activity, a mutanase obtained from a bacterium, especially the Paracoccus mutanolyticus bacterium, the Paenibacillus bacterium or the Streptomyces bacterium, can be used.

In the mixture of glucanohydrolase enzymes, instead of a mixture of mutanase and dextranase, a mixture of chimeric glucanases comprising a dextranase and a mutanase bonded by genetic engineering can also be used.

The glycoside hydrolase enzyme is an enzyme that reduces dimers or trimers of monosaccharides to osidic monomers, i.e. to glucose. It is preferably an amyloglucosidase, more preferably an amylo-α-1,6 glucosidase enzyme. Amyloglucosidase converts oside derivatives into glucose.

The oxidoreductase enzyme is an enzyme that converts glucose into hydrogen peroxide. It is preferably a glucose oxidase, also designated as gluco-oxidase. The gluco-oxidase enzyme converts glucose into D-glucono-delta-lactone and hydrogen peroxide. D-glucono-delta-lactone is an acidity regulator. In the buccal cavity, it is partially converted into gluconic acid.

In the presence of hydrogen peroxide, the peroxidase enzyme converts thiocyanate into hypothiocyanate according to the following reaction:

$$SCN + {H}_{2} {O}_{2} = > OSCN + {H}_{2} O$$

The peroxidase enzyme is preferentially a lactoperoxidase.

Hypothiocyanate, a strong bactericide, limits growth of bacteria in the buccal cavity.

The enzyme complex according to the invention preferably has the following composition (the percentages indicated are percentages by volume):
Invertase: 10 to 30%, preferably 15 to 25%,
Mutanase: 10 to 40%, preferably 15 to 30%,
Dextranase: 1 to 10%, preferably 4 to 7%,
Amyloglucosidase: 20 to 60%, preferably 30 to 50%,
Glucose oxidase: 5 to 20%, preferably 7 to 15%, and
Lactoperoxidase: 1 to 10%, preferably 4 to 7%.

The table below gives an example of a control formulation CET of an enzyme complex with a high amount of amyloglucosidase and without mutanase, and three examples of formulations CE1-3 for the enzyme complex according to the invention. The percentages indicated are percentages by volume.

TABLE 1

| | CET | CE1 | CE2 | CE3 |
|---|---|---|---|---|
| | formu- | formu- | formu- | formu- |
| Compound | lation | lation | lation | lation |
| Invertase | 10% | 10% | 30% | 2% |
| Glucose oxidase | 10% | 10% | 20% | 1% |
| Amyloglucosidase | 70% | 60% | 20% | 3% |
| Lactoperoxidase | 5% | 5% | 10% | 5% |
| Dextranase | 5% | 5% | 10% | 5% |
| Mutanase | 0% | 10% | 10% | 20% |

In clinical trials made on patients over a six-month period by the applicant with the participation of a research laboratory under the responsibility of a doctor of medicine, the formulation CET gave unsatisfactory results. Indeed, compared to the control subjects, in subjects participating in the study for six months and using this formulation, the presence of dental plaque as well as very slight whitening of the tooth enamel (<1 shade) was noticed.

The formulation CE1 gave more satisfactory results than the formulation CET. Indeed, compared to the control subjects, in subjects participating in the study for six months and using this formulation, a lower amount of dental plaque was noticed in the buccal cavity, as well as a clear whitening of the tooth enamel (+1 white shade).

The formulation CE2 gave more satisfactory results than the formulation CET. Indeed, compared to the control subjects, in subjects participating in the study for six months and using this formulation, a lower amount of dental plaque in the buccal cavity was noticed, as well as a clear whitening of the tooth enamel (+1 white shade).

The formulation CE3 gave more satisfactory results than the formulations CE1 and CE2. Indeed, relative to the control subjects, in subjects participating in the study for six months and using this formulation, there was a virtual absence of dental plaque in the buccal cavity, as well as a strong whitening of the tooth enamel (+2 white shades).

The enzyme complex according to the invention may also comprise lysozyme.

The enzyme complex preferably comprises 0.1 to 0.4%, more preferably 0.2% by weight, of lysozyme. It may also preferably comprise 0 to 0.4% by weight of sucrase, preferably an invertase or an isomaltase, more preferably 0.2% by weight.

The enzyme complex according to the invention can be used in a buccal composition for bucco-dental health and enamel whitening.

This buccal composition may, for example, be in the form of a paste, liquid, gel, mouthwash, mist, spray, capsule, tablet or chewing gum.

The buccal composition for oral use according to the invention preferably has the following composition (the enzyme complex is that described previously and the percentages indicated are percentages by weight):
Enzyme complex: 1.4 to 3%, preferably 2.0 to 2.5%,
Sorbitol: 20 to 60%, preferably 30 to 50%,
Xylitol: 1 to 20%, preferably 5 to 10%,
Polysorbate 80:0 to 3%, preferably 1 to 2%,
Polysorbate 20:0 to 3%, preferably 1 to 2%,
Sodium saccharin: 0 to 0.5%, preferably 0.1 to 0.2%,
Colloidal silica: 0 to 5%, preferably 1 to 2%,
Dimethicone: 0 to 3%, preferably 1 to 2%,
Mint flavouring and/or mint essential oils: 0 to 5%, preferably 1 to 2%,
Zinc citrate: 0.1 to 0.5%, preferably 0.1 to 0.3%,
Potassium thiocyanate: 0 to 0.5%, preferably 0.1 to 0.2%,
Preservative: 0 to 0.5%, preferably 0.1 to 0.2%,
Water: sufficient amount to make up 100%.

The preservative is preferably citric acid.

The buccal composition according to the invention may also comprise lactoferrin, preferably 0 to 0.4% by weight, more preferably 0.1%.

It may also comprise hydrogen peroxide, for example from 0 to 0.1%, and violet colorant for a more immediate whitening effect.

The table below gives an example of a control formulation COT of a buccal composition for bucco-dental use with no enzyme complex, and three examples of formulations CO1-3 for the buccal composition according to the invention with the enzyme complex CE3. The percentages indicated are percentages by weight.

TABLE 2

| | Formu- | Formu- | Formu- | Formu- |
|---|---|---|---|---|
| | lation | lation | lation | lation |
| Compound | COT | CO1 | CO2 | CO3 |
| Enzyme complex | 0% | 1.0% | 2.0% | 2.5% |
| Sorbitol | 40% | 40% | 40% | 40% |
| Xylitol | 10% | 10% | 10% | 10% |
| Polysorbate 80 | 1.5% | 1.4% | 1.4% | 1% |
| Polysorbate 20 | 1.5% | 1.4% | 1.4% | 1.4% |
| Sodium saccharin | 0.15% | 0.15% | 0.15% | 0.15% |
| Colloidal silica | 1.5% | 1.5% | 1.5% | 1.5% |
| Dimethicone | 1.5% | 1.5% | 1.5% | 1.5% |
| Mint flavouring and/or | 1.5% | 1.5% | 1.5% | 1.5% |
| mint essential oils | | | | |
| Zinc citrate | 0.2% | 0.2% | 0.2% | 0.2% |
| Potassium thiocyanate | 0.15% | 0.15% | 0.15% | 0.15% |
| Preservative | 0.2% | 0.15% | 0.15% | 0.15% |
| Water | q.s. ad | q.s. ad | q.s. ad | q.s. ad |
| | 100% | 100% | 100% | 100% |

The efficacy of these different formulations has been tested on Streptococcus salivarius, which is from the same Viridans family as Streptococcus mutans, and a Gram-positive cocci bacterium like the latter, also forming a biofilm in the presence of sucrose, and known to have the same sensitivity to antiseptic and antibacterial agents as Streptococcus mutans. The efficacy of these formulations has been compared to a placebo not containing the enzyme complex according to the invention and to a mouthwash solution containing 0.12% chlorhexidine.

In in vitro clinical trials made by the applicant with the participation of another analytical laboratory under the responsibility of a doctor of pharmacy, carried out with the enzyme complex CE3, the formulation COT gave unsatisfactory results. Indeed, relative to placebo vs. formulation COT vs. chlorhexidine, a high amount of Streptococcus salivarius was noticed in the placebo, a still high amount of Streptococcus salivarius was noticed in the formulation COT, and a zero amount was noticed in the formulation based on chlorhexidine.

The formulation CO1 gave more satisfactory results than the formulation COT. Indeed, relative to placebo vs formulation CO1 vs chlorhexidine, a high amount of Streptococcus salivarius was noticed in the placebo, a low amount of Streptococcus salivarius was noticed in the Formulation CO1 and a zero amount in the chlorhexidine-based formulation.

The formulation CO2 gave more satisfactory results than the formulation COT and the formulation CO1. Indeed, relative to placebo vs formulation CO2 vs chlorhexidine, a high amount of Streptococcus salivarius was noticed in the placebo, a zero amount was noticed in the formulation CO2 and a zero amount was noticed in the chlorhexidine-based formulation.

The formulation CO3 gave results as satisfactory as the formulation CO2. Indeed, relative to placebo vs. formulation CO3 vs. chlorhexidine, a high amount of Streptococcus salivarius was noticed in the placebo, a zero amount of Streptococcus salivarius was noticed in the formulation CO3, and a zero amount of Streptococcus salivarius was noticed in the chlorhexidine-based formulation.

Thus, for a buccal composition for bucco-dental use comprising an enzyme complex according to the enzyme formula CE3, although clinical effects could be observed with an amount of enzyme complex of less than 2%, the bactericidal effect against Streptococcus was completely effective with an amount of enzyme complex greater than or equal to 2%.

1. An enzyme complex for use in treatment of dental plaque and bacterial biofilm of the plaque and for natural whitening of tooth enamel, the enzyme complex comprising the following compounds:
a sucrase enzyme,
a mixture of glucanohydrolase enzymes comprising a dextranase, a mutanase having exolytic activity and obtained from a fungus, and a mutanase having endolytic activity and obtained from a bacterium,
a glycoside hydrolase enzyme,
an oxidoreductase enzyme,
a peroxidase enzyme.

2. The enzyme complex according to claim 1, wherein the mixture of glucanohydrolase enzymes comprises a mixture of chimeric glucanases comprising a dextranase and a mutinies bonded by genetic engineering

3. The enzyme complex according to claim 1, wherein the mutanase having exolytic activity is obtained from a Trichoderma Harzianum fungus.

4. The enzyme complex according to claim 1, wherein the mutanase having endolytic activity is obtained from a Paracoccus mutanolyticus, Paenibacillus or Streptomyces bacterium.

5. The enzyme complex according to claim 1, wherein the sucrase enzyme is an isomaltase enzyme or an invertase enzyme.

6. The enzyme complex according to claim 1, wherein the glycoside hydrolase enzyme is an amyloglucosidase enzyme.

7. The enzyme complex according to claim 1, wherein the oxidoreductase enzyme is a glucose oxidase.

8. The enzyme complex according to claim 1, wherein the peroxidase enzyme is a lactoperoxidase.

9. The enzyme complex according to claim 1, comprising the following compounds:
a sucrase,
a dextranase,
a mixture of mutanases comprising a mutanase having exolytic activity and obtained from a fungus, and a mutanase having endolytic activity and obtained from a bacterium,
an amyloglucosidase,
a glucose oxidase, and
a lactoperoxidase.

10. The enzyme complex according to claim 8, comprising the following compounds, the percentages indicated being percentages by volume:
10 to 30% of a sucrase,
1 to 10% of dextranase,
10 to 40% of a mixture comprising a mutanase having exolytic activity and obtained from a fungus and a mutanase having endolytic activity and obtained from a bacterium,
20 to 60% of an amyloglucosidase,
5 to 20% of glucose oxidase, and
1 to 10% of lactoperoxidase.

11. A buccal composition for use in the treatment of dental plaque and bacterial biofilm of the plaque and the natural whitening of tooth enamel, comprising an enzyme complex according to claim 1.

12. The buccal composition according to claim 11, comprising from 1.4 to 3% of enzyme complex.

13. The buccal composition according to claim 11, further comprising lactoferrin.

14. The buccal composition according to claim 11, further comprising:
20 to 60% of sorbitol,
1 to 20% of xylitol,
0 to 3% of polysorbate 80,
0 to 3% of polysorbate 20,
0 to 0.5% of sodium saccharin,
0 to 5% of colloidal silica,
0 to 3% of dimethicone,
0 to 5% of mint flavouring and/or mint essential oils,
0.1 to 0.5% of zinc citrate,
0 to 0.5% of potassium thiocyanate,
0 to 0.5% of preservative,
water, in sufficient quantity to make up 100%.

15. The buccal composition according to claim 11, in the form of a paste, liquid, gel, mouthwash, mist, spray, capsule, tablet or chewing gum.

16. The enzyme complex according to claim 6, wherein the amyloglucosidase enzyme is an amyl-α-1,6 glucosidase.

17. The enzyme complex according to claim 9, wherein the sucrase is an invertase or an isomaltase, and the amyloglucosidase is an amylo-α-1,6 glucosidase.

18. The enzyme complex according to claim 10, comprising the following compounds, the percentages indicated being percentages by volume:
15 to 25% of a sucrase,
4 to 7% of dextranase,
15 to 30% of a mixture comprising a mutanase having exolytic activity and obtained from a fungus and a mutanase having endolytic activity and obtained from a bacterium,
30 to 50% of an amyloglucosidase,
7 to 15% of glucose oxidase, and
4 to 7% of lactoperoxidase.

19. The buccal composition according to claim 12, comprising from 2.0 to 2.5% of enzyme complex.

20. The buccal composition according to claim 14, comprising:
30 to 50% of sorbitol,
5 to 10% of xylitol,
1 to 2% of polysorbate 80,
1 to 2% of polysorbate 20,
0.1 to 0.2% of sodium saccharin,
1 to 2% of colloidal silica,
1 to 2% of dimethicone,
1 to 2% of mint flavouring and/or mint essential oils,
0.1 to 0.3% of zinc citrate,
0.1 to 0.2% of potassium thiocyanate,
0.1 to 0.2% of preservative.
